(19) United States
(12) Patent Application Publication
(10) Pub. No.: US 2002/0045266 A1
(43) Pub. Date: Apr. 18, 2002

(54) Method for determining the structure of an active member of a chemical library

(76) Inventor: Hicham Fenniri, West Lafayette, IN (US)

Correspondence Address: Bradford G. Addison, Maginot, Addison & Moore, Bank One Center/Tower, 111 Monument Circle, Suite 3000, Indianapolis, IN, 46204-5130, US

(21) Appl. No.: 09/778,374

(22) Filed: Feb. 7, 2001

Related U.S. Application Data
(60) Provisional application No. 60/180,939, filed on Feb. 8, 2000.

(51) Int. Cl. G01N 33/545; B01J 19/00

(52) U.S. Cl. 436/85; 436/532; 436/171; 436/86

(57) ABSTRACT

A method for determining the structure of an active member of a chemical library is disclosed.

This non-provisional U.S. patent application claims the benefit of U.S. provisional patent application Ser. No. 60/180,939 filed on Feb. 8, 2000.

### BACKGROUND OF THE INVENTION

The present invention generally relates to a method for determining the structure of an active member of a chemical library. The present invention particularly relates to a method for determining the primary structure of an active compound of a combinatorial library.

Combinatorial synthetic methods allow for the preparation of large arrays of compounds as mixtures or individual entities. A technique known as split synthesis has proven to be ideal in maximizing the number of compounds generated per synthetic step. While the composition of the chemical or combinatorial libraries produced using this method is predictable with a high level of confidence, the structural elucidation of members of the libraries which possess a desired activity (e.g. binding to a particular receptor) is quite challenging. Several strategies to unravel the chemical nature or structure of an active member of a combinatorial library subsequent to an activity assay have been developed. For example, several tactics based on encoding methodologies or deconvolutive strategies have been employed. However, these encoding and deconvolutive techniques tend to be time consuming and expensive which increases the cost of developing useful compounds, such as pharmaceuticals.

Therefore, in light of the above discussion, it is apparent that what is needed is a strategy for the structural elucidation of active members of combinatorial libraries that addresses one or more of the above discussed drawbacks.

### SUMMARY OF THE INVENTION

In accordance with one embodiment of the present invention, there is provided a method for determining the primary structure of a first compound which is bound to a solid support matrix. The method includes (a) reacting a first building block of the first compound with the first solid support matrix so that the first building block is bound to the first solid support matrix, (b) subjecting the first solid support matrix to a spectroscopic technique so as to generate spectrographic data of the first solid support matrix, (c) determining a chemical composition of the first solid support matrix the first building block is bound to based upon the data generated by the spectrographic technique, and (d) determining the chemical identity of the first building block based upon the chemical composition of the first solid support matrix.

In accordance with another embodiment of the present invention, there is provided a method of screening a combinatorial library which includes (i) a first solid support matrix, (ii) a second solid support matrix, (iii) a first compound having a building block thereof directly chemically bound to the first solid support matrix, and (iv) a second compound having a building block thereof directly chemically bound to the second solid support matrix. The first compound has a primary structure which is different from the primary structure of the second compound and the first solid support matrix has a chemical composition which is spectroscopically distinct from a chemical composition of the second solid support matrix. The method includes (a) subjecting the first solid support matrix to a spectroscopic technique so as to generate spectrographic data of the first solid support matrix, (b) utilizing the spectrographic data to distinguish the first solid support matrix from the second solid support matrix, and (c) determining the chemical identity of the building block of the first compound which is directly chemically bound to the first solid support matrix based upon the spectroscopically distinct chemical composition of the first solid support matrix.

In accordance with still another embodiment of the present invention there is provided a method of screening a combinatorial library which includes (i) a first bead, (ii) a second bead, (iii) a first amino acid oligomer having an amino acid located in a first position, the amino acid located in the first position being directly chemically bound to the first bead, and (iv) a second amino acid oligomer chemically bound to the second bead, wherein (i) the first amino acid oligomer has a primary structure which is different from the primary structure of the second amino acid oligomer and (ii) the first bead has a chemical composition which is spectroscopically distinct from a chemical composition of the second bead. The method includes the steps of (a) subjecting the first bead to a spectroscopic technique so as to generate spectrographic data of the first bead, (b) utilizing the spectrographic data to distinguish the first bead from the second bead, and (c) determining the chemical identity of the amino acid of the first amino acid oligomer which is located in the first position based upon the spectroscopically distinct chemical composition of the first bead.

It is therefore an object of the present invention to provide a new and useful method for determining the structure of an active member of a chemical library.

It is another object of the present invention to provide improved method for determining the structure of an active member of a chemical library.

It is still another object of the present invention to provide a method for determining the structure of an active member of a chemical library which includes a non-invasive screening technique to determine the identity of a building block located in a randomized first position.

It is another object of the present invention to provide a method for determining the structure of an active member of a chemical library which is inexpensive and does not require the development of any complicated encoding chemistry.

The above and other objects, features, and advantages of the present invention will become apparent from the following description and attached drawings.

### BRIEF DESCRIPTION OF THE DRAWINGS

The file of this patent contains at least one drawing executed in color. Copies of this patent with color drawings(s) will be provided by the Patent and Trademark Office upon request and payment of the necessary fee.

FIG. 1 is a schematic representation of a combinatorial synthetic methodology for generating a combinatorial library of compounds;

FIG. 1A is a schematic representation of a methodology for determining the structure of an active member of the combinatorial library generated by the methodology of FIG. 1;

FIG. 2 is a schematic representation of a combinatorial synthetic methodology for generating a combinatorial library of compounds which incorporates the features of the present invention therein;

FIG. 2A is a is a schematic representation of a methodology for determining the structure of an active member of the combinatorial library generated by the methodology of FIG. 2 which incorporates the features of the present invention therein;

FIG. 3 is another schematic representation of a combinatorial synthetic methodology for generating a combinatorial library of compounds which incorporates the features of the present invention therein;

FIG. 4 panels A and C show Raman spectrum of TentaGel beads illustrating vibrations specific to the polyethyleneglycol (PEG) component thereof as well as the fingerprint transitions of polystyrene (PS), while panels B, D, E, and F show images of TentaGel-S—OH beads and/or polystyrene beads derived from Raman data;

FIG. 5 panels B, E, and I show a white light image of various beads, while panels A, C, D, F, G, and H show NIR-Raman imaging of various beads;

FIG. 6 is an enlarged view of panel I of FIG. 5;

FIG. 7 is a single 4-Bromo-PS bead near IR-Raman spectrum;

FIG. 8 is a single PEG crosslinked-PS bead near IR-Raman spectrum;

FIG. 9 is a single Amino-PEGA bead near IR-Raman spectrum;

FIG. 10 is a single HMBA-SPAR 50 bead near IR-Raman spectrum;

FIG. 11 is a single Amino-PEGA bead near IR-Raman spectrum;

FIG. 12 is a single SPAR 50 bead near IR-Raman spectrum;

FIG. 13 is a single SPAR 50 bead near IR-Raman spectrum;

FIG. 14 is a single SPAR 50 bead near IR-Raman spectrum;

FIG. 15 is a single Carboxy-PS bead near IR-Raman spectrum;

FIG. 16 is a single Carboxy-PS bead near IR-Raman spectrum;

FIG. 17 is a single HMBA-SPAR 50 bead near IR-Raman spectrum;

FIG. 18 is a single Amino-PEGA bead near IR-Raman spectrum;

FIG. 19 is a single Carboxy-PS bead near IR-Raman spectrum;

FIG. 20 is a single HMBA-SPAR 50 bead near IR-Raman spectrum;

FIG. 21 is a single 4-Bromo-PS bead near IR-Raman spectrum;

FIG. 22 is single Amino-PEGA bead near IR-Raman spectrum;

FIG. 23 is a single PEG crosslinked-PS bead near IR-Raman spectrum;

FIG. 24 is a single HMBA-SPAR 50 bead near IR-Raman spectrum;

FIG. 25 is a single Carboxy-PS bead near IR-Raman spectrum;

FIG. 26 is a single PEG crosslinked-PS bead near IR-Raman spectrum; and

FIG. 27. is a table summarizing the conditions for bead synthesis.

### DETAILED DESCRIPTION OF THE PREFERRED EMBODIMENT

While the invention is susceptible to various modifications and alternative forms, a specific embodiment thereof has been shown by way of example in the drawings and will herein be described in detail. It should be understood, however, that there is no intent to limit the invention to the particular form disclosed, but on the contrary, the intention is to cover all modifications, equivalents, and alternatives falling within the spirit and scope of the invention as defined by the appended claims.

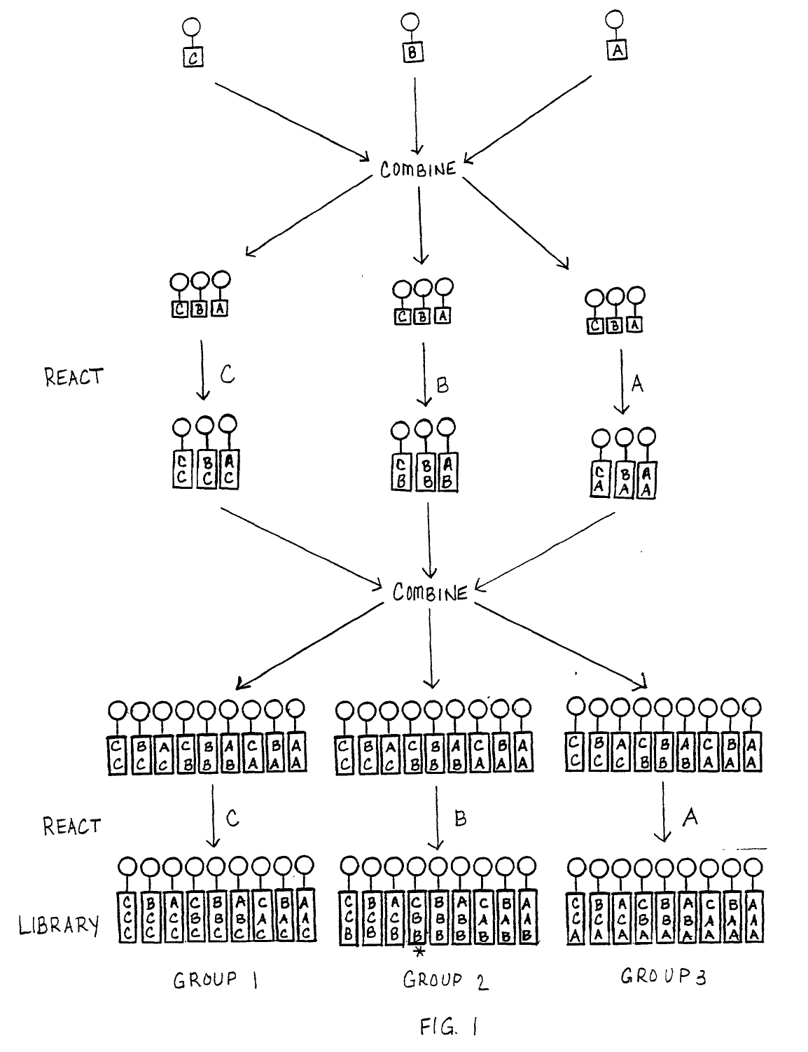

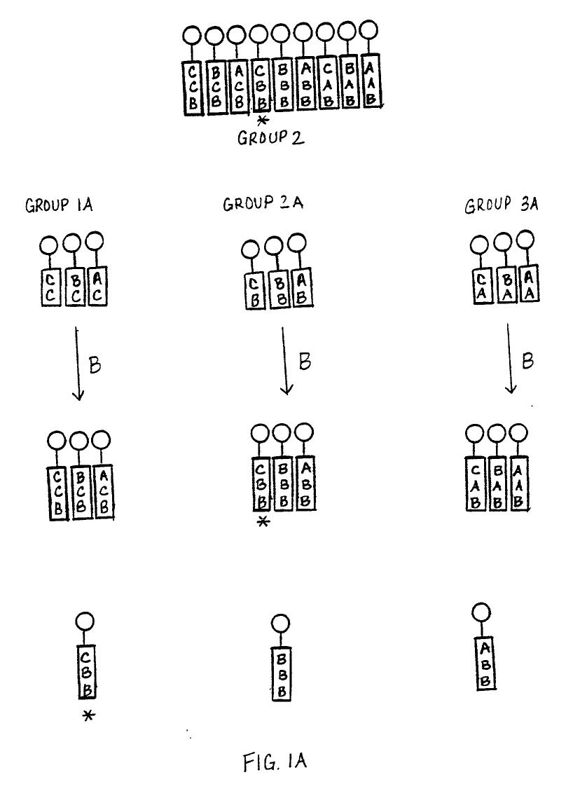

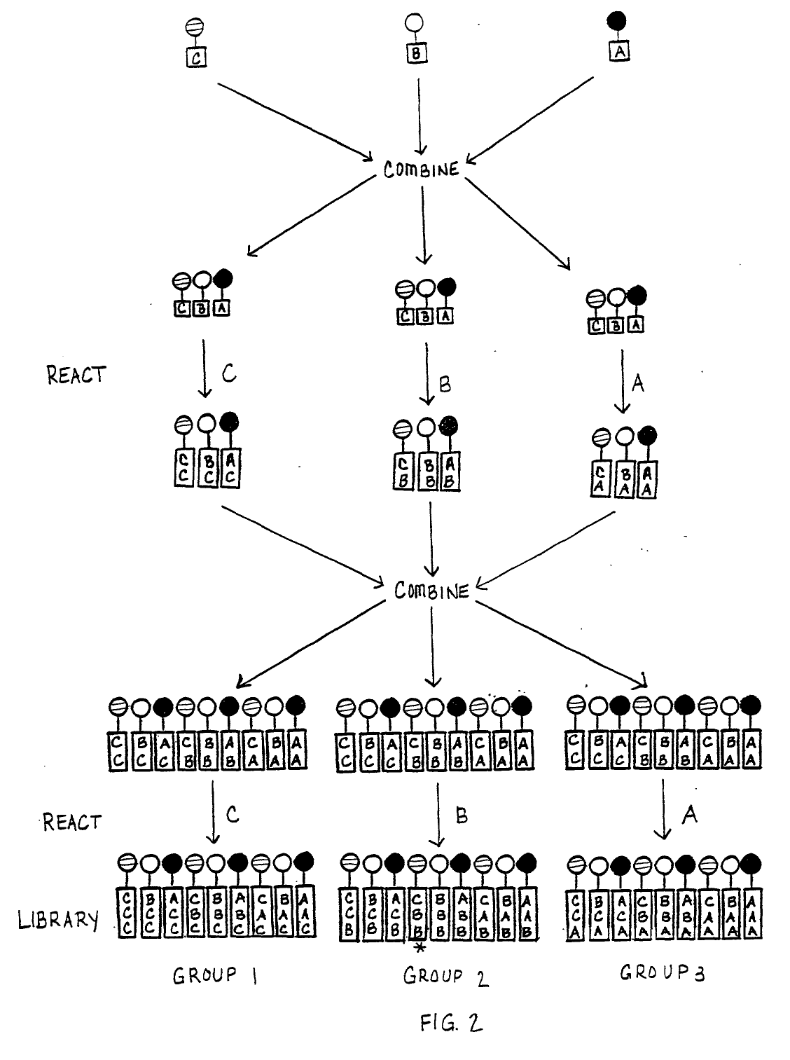

Referring now to FIG. 1, there is shown a schematic representation of a combinatorial synthetic methodology for generating a combinatorial library of compounds. What is meant herein by a combinatorial library is a group of compounds generated by a combinatorial synthetic methodology. For example, one way of generating a combinatorial library is described in A. Furka, L. K. Hamaker, M. L. Peterson in Combinatorial Chemistry: A Practical Approach, (Ed.: H. Fenniri), Oxford University Press, Oxford, 2000, which is incorporated herein by reference. Note that in FIG. 1 the circles (∘) represent a solid support matrix in the form of a bead, and each letter disposed in a rectangle, e.g. □, represents a building block of a compound which is synthesized with the combinatorial synthetic methodology. Note that the term “building block” as used herein refers to any molecule making up a compound. For illustrative purposes the compounds generated utilizing the combinatorial synthetic methodology depicted in FIG. 1 are amino acid oligomers (e.g. a tripeptide), and thus each letter in a rectangle represents one amino acid, and one amino acid represents one building block of the compound. It should be understood that the term “oligomer” as used herein includes compounds having two or more building block units. It should also be understood that the letter in the rectangle does not represent any particular amino acid (e.g. “A” represent one amino acid while “B” represents another amino acid). Furthermore, as discussed in greater detail below in reference to FIGS. 2 and 2A, the present invention is not limited to determining the structure of amino acid oligomers. On the contrary, it is contemplated that the present invention can be utilized to determine the structure of a large number of other types of compounds, i.e. any compound which includes a plurality of chemically bound building block molecules. For example, other types of compounds which the present invention can be utilized to determine the structure thereof include, but is not limited to, nucleic acids (e.g. oligonucleotides) and carbohydrates (e.g. oligosaccharides).

The methodology illustrated in FIG. 1 utilizes the portioning-mixing (split-mix) procedure to generate the combinatorial library. In particular, initially the solid support matrix, hereinafter referred to as the beads, are divided into 3 equal portions and one amino acid is covalently bound to each portion of beads via well known chemical methods which are not discussed in detail herein. This step results in 3 groups of beads, with one group being covalently bound to amino acid “C”, one group being covalently bound to amino acid “B”, and one group be covalently bound to amino acid “A”. These 3 groups are then combined and once again divided into 3 equal portions or groups with each group containing (i) beads covalently bound to amino acid “C”, (ii) beads covalently bound to amino acid “B”, and (iii) beads covalently bound to amino acid “A”. Each group is then reacted with one amino acid such that the amino acid covalently bound directly to a bead is also covalently bound to an amino acid to yield a dipeptide bound to each bead. In particular, as clearly shown in FIG. 1, the group of beads reacted with amino acid “C” yields a group of beads containing (i) beads covalently bound to the dipeptide “C-C”, (ii) beads covalently bound to the dipeptide “B-C”, and (iii) beads covalently bound to the dipeptide “A-C”. The group of beads reacted with amino acid “B” yields a group of beads containing (i) beads covalently bound to the dipeptide “C-B”, (ii) beads covalently bound to the dipeptide “B-B”, and (iii) beads covalently bound to the dipeptide “A-B”. In a similar fashion, the group of beads reacted with amino acid “A” yields a group of beads containing (i) beads covalently bound to the dipeptide “C-A”, (ii) beads covalently bound to the dipeptide “B-A”, and (iii) beads covalently bound to the dipeptide “A-A”. All 3 groups of beads covalently bound to a dipeptide are once again combined and then divided into 3 equal portions, with each group containing beads covalently bound to a dipeptide as shown in FIG. 1. Once again, each of these groups is reacted with one amino acid, i.e. “C”, “B”, or “A”, such that each bead is now covalently bound to a tripeptide. At this point there are 3 groups of beads, with each group containing nine different tripeptides, for a total of 27 different peptides. (Note that each tripeptide is covalently bound to a bead.) This pool of 27 different tripeptides, divided into 3 separate groups of 9 distinct tripeptides per group, is the combinatorial library of compounds for this particular example. Note that hereinafter the amino acid directly bound to the bead will be referred to as being in position 1, the next amino acid will be referred to as being in position 2, and the last amino acid will be referred to as being in position 3. For example, the following schematic representation of a tripeptide covalently bound to a bead 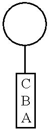 has the amino acid “C” in the first position, the amino acid “B” in the second position, and the amino acid “A” in the third position. This designation of building block positions will be utilized through out the present disclosure. Also note that the groups making up the aforementioned combinatorial library will be referred to as group 1, group 2, and group 3 (see FIG. 1). Further note that, based upon the combinatorial synthetic methodology described above, without any further chemical analysis, it is already known that (i) all of the tripeptides of group 1 must have the amino acid “C” in the third position, (ii) all of the tripeptides of group 2 must have the amino acid “B” in the third position, and (iii) all of the tripeptides of group 3 must have the amino acid “A” in the third position.

Still referring to FIG. 1, each group (i.e. group 1, 2, and 3) of the above described combinatorial library is screened to determine whether any of the 27 tripeptides contained therein possess a desired activity. The combinatorial library can be screened with any well known technique including, but not limited to, determining whether any of the tripeptides bind to, or inhibit another compound from binding to, a receptor with a fluorescent label. For example, of the 3 groups which make up the combinatorial library of FIG. 1 assume that upon screening group 2 tests positive for a desired activity. Therefore, group 2 must contain a tripeptide which possess a desired activity.

Based upon the combinatorial synthetic methodology described above it is known that group 2 contains the bead bound tripeptides shown in FIG. 1A and that they all have the amino acid “B” in the third position, however, it is not known which particular peptide contained in group 2 possess the desired activity. For illustrative purposes assume that the tripeptide having the * adjacent thereto is the active peptide. In order to determine the primary structure of the active tripeptide contained in group 2 the amino acids in the first position and the second position must also be determined. In order to determine the amino acids in the first and second positions the following additional steps must be taken. First, 3 groups of dipeptides covalently bound to beads are generated. In particular, as clearly shown in FIG. 1A, the first group (labeled as group 1A) contains (i) beads covalently bound to the dipeptide “C-C”, (ii) beads covalently bound to the dipeptide “B-C”, and (iii) beads covalently bound to the dipeptide “A-C”. The second group (labeled as group 2A) contains (i) beads covalently bound to the dipeptide “C-B”, (ii) beads covalently bound to the dipeptide “B-B”, and (iii) beads covalently bound to the dipeptide “A-B”. The third group (labeled as group 3A) contains (i) beads covalently bound to the dipeptide “C-A”, (ii) beads covalently bound to the dipeptide “B-A”, and (iii) beads covalently bound to the dipeptide “A-A”. Since all of the tripeptides in group 2 of the combinatorial library end in amino acid “B” (see FIG. 1), the amino acid in position 3 of the active tripeptide is already known, i.e. it must be amino acid “B”. Therefore, each of these groups (i.e. group 1A, 2A, and 3A) is reacted with amino acid “B” such that each bead is now covalently bound to a tripeptide with amino acid “B” in the third position as shown in FIG. 1A. It should also be appreciated that the amino acid in the second position for each tripeptide is also known, i.e. the amino acid in the second position of the tripeptides synthesized from group 1A is “C”, the amino acid in the second position of the tripeptides synthesized from group 2A is “B”, and the amino acid in the second position of the tripeptides synthesized from group 3A is “A”. At this point there are 3 groups of bead bound tripeptides, with each group containing 3 distinct tripeptides for a total of 9 tripeptides to be screened. Each group of tripeptides is then screened to determine which group contains the tripeptide that possess the desired activity. As indicated by the * in FIG. 1A, the group containing the tripeptide having the amino acid “C” in the first position, the amino acid “B” in the second position, and the amino acid “B” in the third position will screen positive for the desired activity. As previously discussed, based upon the synthesis methodology utilized to prepare the compounds, the amino acids in the second and third positions of the tripeptides in this group are already known, i.e. the amino acid in the second position must be a “B” and the amino acid in the third position must also be a “B”. Thus the amino acid in the first position of the active tripeptide must be determined. In order to do this each tripeptide contained in the “active group” (i.e. the group derived from group 2A of FIG. 1A) is synthesized and screened separately, i.e. one tripeptide with the amino acid “C” in the first position is synthesized and screened, one tripeptide with the amino acid “B” in the first position is synthesized and screened, and one tripeptide with the amino acid “A” in the first position is synthesized and screened. As shown by the * in FIG. 1A the tripeptide having the amino acid “C” in the first position will screen positive for the desired activity. Therefore, since the amino acid in the first position of the active tripeptide is now known, i.e. “C”, and the amino acids in the second and third positions, i.e. “B”, are already known, the entire primary structure or sequence of building blocks, i.e. amino acids, is known for the active tripeptide. Thus the above described procedure results in a complete description of the covalent connections of the tripeptide.

While the above described procedure does result in the determination of the primary structure of an active compound, it is rather tedious and time consuming which can add to the expense of performing such an analysis.

Now referring to FIGS. 2 and 2A, there is shown an exemplary schematic representation of a combinatorial synthetic methodology for generating a combinatorial library of compounds which incorporates the features of the present invention therein. As will become apparent from the following discussion, the methodology and associated solid support matrix of the present invention provides an enhanced ability to efficiently determine the primary structure of a compound having a desired activity.

The exemplary schematic representation shown in FIG. 2 utilizes the same portioning-mixing (split-mix) procedure to generate the combinatorial library as discussed above and therefore will not be described in as great a detail hereinafter. However, it should be appreciated that it is contemplated that other procedures to generate a chemical library can be utilized with the present invention. Also note that, as will be discussed in greater detail below, in FIG. 2 and 2A the circles ( [symbol] , ∘, and ●) represent 3 spectroscopically distinct solid support matrices each in the form of a bead, however the present invention is not limited to a bead configuration as other configurations can also be utilized in the present invention. Moreover, each letter disposed in a rectangle, e.g. □, represents a building block of a compound which is synthesized with the exemplary combinatorial synthetic methodology. For illustrative purposes, like FIG. 1, the compounds generated utilizing the combinatorial synthetic methodology depicted in FIGS. 2 and 2A are amino acid oligomers (e.g. a tripeptide), and thus each letter in a rectangle represents one amino acid. However, as previously mentioned, the present invention is not limited to amino acid oligomers, on the contrary it is contemplated that the present invention can be utilized to determine the primary structure of a with wide variety of compounds.

Utilizing the methodology illustrated in FIG. 2 the combinatorial library is generated by initially providing 3 groups of spectroscopically distinct beads with each group containing about the same number of beads. It should be understood that initially each group only contains one type of spectroscopically distinct bead. As mentioned above, the first group of spectroscopically distinct beads is indicated by the symbol [symbol] , the second group of spectroscopically distinct beads is indicated by the symbol ∘, and the third group of spectroscopically distinct beads is indicated by the symbol ●. What is meant herein by the phrase “spectroscopically distinct” is that each solid support matrix (e.g. each bead) is structurally or chemically encoded in a manner such that when the beads are subjected to a spectrographic technique, data generated from that spectrographic technique allows each solid support matrix to be distinguished from one another. For example, one way of chemically encoding each solid support matrix is to “tag” each solid support matrix with a spectroscopically distinct chemical group. By tagging each solid support matrix with a spectroscopically distinct chemical group the data generated from the spectroscopic technique allows the determination of which solid support matrix includes which distinct “tag” and thereby allows the solid support matrices to be distinguished from one another. In other words, the data generated by the spectroscopic technique allows the determination of the chemical composition of one or more of the solid support matrices and thus allows each solid support matrix to be distinguished from each other.

As shown in FIG. 2, once the spectroscopically distinct beads have been provided in the above described manner one amino acid is covalently bound to each portion of beads via well known chemical methods. This step results in 3 groups of beads, with one group being covalently bound to amino acid “C”, one group being covalently bound to amino acid “B”, and one group being covalently bound to amino acid “A”. At this point it should be appreciated that the amino acid located in the first position of any compound subsequently synthesized can easily be determined at any point in the process by simply subjecting the solid support matrix (i.e. the beads) to a spectrographic technique and determining which solid support matrix the compound is attached to. For example, as illustrated in FIG. 2, if a compound is covalently or otherwise bound to, or associated with, a bead spectroscopically determined to be from the first group of beads (indicated by the symbol [symbol] ), then the identity of the amino acid in the first position is automatically known, i.e. the amino acid in the first position must be “C”. In a similar manner, if a compound is bound to, or associated with, a bead spectroscopically determined to be from the second group of beads (indicated by the symbol ∘), then the identity of the amino acid in the first position must be “B”. Finally, if a compound is bound to, or associated with, a bead spectroscopically determined to be from the third group of beads (indicated by the symbol ●), then the identity of the amino acid in the first position must be “A”.

Once the amino acid in the first position is attached to the beads in the above described manner, the beads are then subjected to the same portioning-mixing (split-mix) procedure described in reference to FIG. 1 to generate the combinatorial library shown in FIG. 2. In particular, similar to the combinatorial library shown in FIG. 1, the combinatorial library of FIG. 2 contains 3 groups of beads, with each group containing nine different tripeptides, for a total of 27 different peptides. Note that the groups making up the aforementioned combinatorial library of FIG. 2 will also be referred to as group 1, group 2, and group 3. Also note that, based upon the combinatorial synthetic methodology utilized to generate the combinatorial library of FIG. 2, without any further chemical analysis, it is already known that (i) all of the tripeptides of group 1 must have the amino acid “C” in the third position, (ii) all of the tripeptides of group 2 must have the amino acid “B” in the third position, and (iii) all of the tripeptides of group 3 must have the amino acid “A” in the third position. Further note that each group contains all three different types of spectroscopically distinct beads.

As with the combinatorial library of FIG. 1, the combinatorial library of FIG. 2 is screened to determine if any of the tripeptides contained therein possess a desired activity. As previously discussed, the combinatorial library can be screened with any well known technique including, but not limited to, determining whether any of the tripeptides bind to, or inhibit another compound from binding to, a receptor with a fluorescent label. Note that it is preferable that the library be screened prior to cleaving the compound from the solid support matrix. However, it is contemplated that the screening can take place subsequent to cleaving the compound from the solid support matrix. Assume that, like the combinatorial library of FIG. 1, group 2 of FIG. 2 tests positive for a desired activity and that the tripeptide having the * adjacent thereto is the active peptide. Based upon the combinatorial synthetic methodology utilized to generate the library, this group will contain the bead bound tripeptides shown in group 2 of FIG. 2. It is further known that all of the tripeptides in group 2 have the amino acid “B” in the third position. Therefore, one needs to determine the identity of the amino acids in the first and second positions to ascertain the entire primary structure of the active tripeptide. In order to do this utilizing the present invention, beads attached to a tripeptide exhibiting a desired activity are selected and then subjected to a spectrographic technique. (Note that the selected beads do not have to be physically removed or separated from the other beads for the present invention to function properly.) For example, one way of selecting beads having a compound attached thereto which expresses a desired activity is to utilize a receptor having an attached fluorescent label in a receptor binding assay. In particular, the desired activity screened for is the ability of the tripeptide to bind to the fluorescently labeled receptor. Using this example, the beads having the tripeptide attached thereto which possess the desired activity, i.e. the ability to bind to the fluorescently labeled receptor, are easily selected based upon their fluorescence. Once selected, the beads attached to the active tripeptide are subjected to a spectroscopic technique (obviously the spectroscopic technique utilized is one which is not interfered with by the fluorescence of the beads) to determine the type of bead (e.g. [symbol] , ∘, or ●) the active tripeptide is attached to. Once the type of bead the active tripeptide is attached to is known, the identity of the amino acid in the first position is also known. Since the identity of the amino acid in the third position is already known, all that remains to determine the entire primary sequence of the compound (i.e. the tripeptide) is the identity of the amino acid in the second position.

The determination of the identity of the amino acid in the second position is easily accomplished. In particular once again assume that for illustrative purposes the tripeptide having the * adjacent thereto in group 2 is the active compound (see FIG. 2). Once the beads attached to the active tripeptide have been selected based upon their fluorescence, and then subjected to a spectrographic technique in order to determine their type, the identity of the amino acids in the first and third positions are known. Thus, as shown in FIG. 2A, all that needs to be done is to separately synthesize three tripeptides with each one having an alternate amino acid in position 2, i.e. amino acid “C”, “B”, or “A”. These three tripeptides are then separately screened to determine which one possess the desired activity. Once it is determined which of the three tripeptides has the desired activity the identity of the amino acid located in the second position is known. Therefore, the identity of the amino acid in all three positions is known, and the primary structure of the compound has been determined.

Based upon the above discussion it should be appreciated that the present invention operates through the iterative identification of the building blocks located in the first (i.e. position 1) and last randomized positions of active members of combinatorial libraries generated through split synthesis. The identification of the building block located in the last position (e.g. position 3 in the above described tripeptide example) is readily obtained from group screening after the last coupling of the split synthesis, while the first position can be encoded by the unique spectroscopic characteristic or vibrational fingerprint of the solid support matrix (e.g. beads) used. Once the building blocks located in the first and last positions are identified, the building blocks located in the second and second to last positions are then subjected to a deconvolution process in order to determine their identity. Remarkably, the present invention dramatically simplifies the synthetic and screening efforts required to investigate compounds having a desired activity as compared to other methodologies.

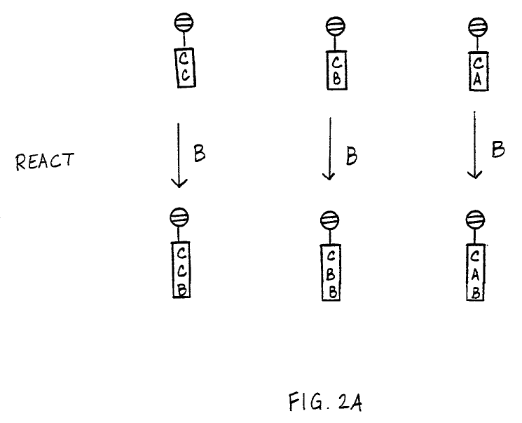

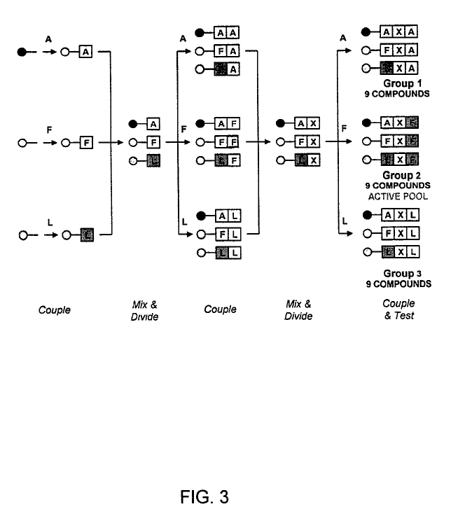

Now referring to FIG. 3, there is shown another exemplary schematic representation of a combinatorial synthetic methodology for generating a combinatorial library of compounds which incorporates the features of the present invention therein. FIG. 3 is similar to FIG. 2 but illustrates the methodology of the present invention in a more generalized manner as compared to FIG. 2. In FIG. 3 the three spectroscopically distinguishable beads are depicted as black, white, and gray spheres. The bead depicted as a black sphere is used to encode the building block “A” in the first position, the bead depicted as a white sphere is used to encode the building block “F” in the first position, and the bead depicted as a gray sphere is used to encode the building block “L” in the first position. X denotes any of the building blocks “A”, “F” or “L”. The identity of the building block in the last position of an active member of the library is revealed by group assay after the last step of the split synthesis, while the identity of the building block in the first position is unveiled, as discussed above, by subjecting the beads to a spectrographic technique, e.g. as will be discussed in greater detail below multispectral imaging of the beads attached to an active compound. The gray shaded rectangles highlight the building blocks required for the desired activity.

FIG. 3 outlines the analysis of an exemplary 27 member combinatorial library generated through split synthesis. The last step of this process generates 3 groups with each group containing 9 compounds. Screening of each of the groups separately identifies the best third position. As discussed above, each bead encodes and thus identifies the first randomized building block. The analysis of the library operates through the identification of the last (group assay) and first (spectroscopically encoded beads) randomized positions. This process is then repeated iteratively for the remaining unidentified positions until the entire sequence of the active library member(s) is unveiled.

TABLE 1

| | | | | | | | Libraries to be |
|---|---|---|---|---|---|---|---|
| L^{[a]} | N^{[b]} | M^{[c]} | S₁^{[d]} | R₁^{[e]} | S₂^{[f]} | R₂^{[g]} | synthesized^{[h]} |
| 3 | 10 20 | 1 × 10³ 8 × 10³ | 30 60 | 33 133 | 42 (S₁ + 12) 82 (S₁ × 22) | 24 98 | 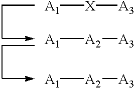 |
| 4 | 10 20 | 1 × 10⁴ 1.6 × 10⁵ | 40 80 | 250 2,000 | 62 (S₁ + 22) 122 (S₁ + 42) | 161 1,312 | 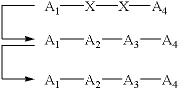 |
| 5 | 10 20 | 1 × 10⁵ 3.2 × 10⁶ | 50 100 | 2,000 32,000 | 96 (S₁ + 32 + 14) 186 (S₁ + 62 + 24) | 1,042 17,204 | 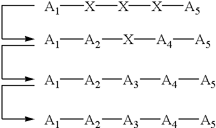 |
| 6 | 10 20 | 1 × 10⁶ 64 × 10⁶ | 60 120 | 16,667 533,333 | 126 (S₁ + 42 + 24) 246 (S₁ + 82 + 44) | 7,937 260,163 | 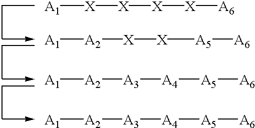 |

^{[a]}L is the number of synthetic steps per library member, L is also the number of building blocks per library member.
^{[b]}N is the number of building blocks.
^{[c]}M = N^{L} is the library size using split synthesis.
^{[d]}S₁ = N × L is the number of synthetic steps for the preparation of the library using the split synthesis method.
^{[e]}R₁ = M × (S₁)⁻¹ corresponds to the number of compounds generated per synthetic step. [f] S₂ is the total number of synthetic steps for library and sub-library synthesis, and for full identification of an active member of the library using the methodology of the present invention.
^{[g]}R₂ = M × (S₂)⁻¹ is the number of compounds synthesized and screened per synthetic step. As indicated by this ratio the larger the library the fewer the number of steps for its preparation and for the identification of the active members.
^{[h]}General formula of the libraries and sub-libraries to be synthesized using split synthesis and the present invention for complete chemical identification of an active member of the library. X denotes randomized positions to be identified in subsequent sub-libraries. Aₙ denotes randomized positions to be identified at that step. Bold Aₙ denotes positions identified in preceding sub-library steps.

In reference to Table 1, the combinatorial libraries vary in number of steps from 3 to 6 and utilize 10 to 20 building blocks. The number of steps for the preparation of a library using the split synthesis (column 4) varies linearly while the size of the library increases exponentially (column 3). The number of compounds synthesized per chemical step (column 5) increases rapidly as the library size increases, thereby highlighting the strength of the split synthesis method. Likewise, columns 6 and 7 show a similar trend except that in this case the ratio of compounds synthesized to the number of chemical steps includes the steps required by the present invention and hence the full identification (i.e. primary structure) of the active member of the library. For instance, utilizing the present invention for the synthesis, screening, and full identification of an active member of a 64-million member library would barely double the number of chemical steps required for the synthesis of the library using the split synthesis method (246 versus 120). This clearly illustrates the advantage of the present invention. The last column of Table 1 shows the general formula of the libraries and sub-libraries to be synthesized with three to six building blocks or chemical transformations per member using split synthesis and the present invention.

With respect to subjecting a solid support matrix to a spectroscopic technique, a Near Infrared Raman Imaging (NIRIM) instrument was used as a tool for the simultaneous identification of beads of various chemical composition (A. D. Gift, J. Ma, K. S. Haber, B. L. McClain, D. Ben-Amotz, J. Raman Spectrosc. 1999, 30, 757-765, incorporated herein by reference). The NIRIM uses fiber bundle image compression (FIC) technology to simultaneously collect a 3-D Raman spectral imaging data cube (λ-x-y) containing an optical spectrum (λ) at each spatial location (x-y) of a globally illuminated area (J. Ma, D. Ben-Amotz, Applied Spectrosc. 1997, 51, 1845-1848, incorporated herein by reference). It should be noted that this is a real-time imaging technique as opposed to other step-scan methods, which require much longer time to generate an image of the sample.

The NIRIM instrument uses near infrared (NIR) external cavity narrow band, 400 mW, 785 nm diode laser (SDL-8630), which maximizes resolution and reduces sample fluorescence interference. The charge coupled device (CCD) detector (Princeton instruments LN/CCD-1024 EHRB) has a deep depletion, back illuminated chip which is NIR anti-reflection coated and roughened to virtually eliminate etaloning artifacts (quantum efficiency of 85% at 785 nm and 20% at 1050 nm). The NIRIM also uses a Kaiser Holoscop Imaging spectrograph with an input lens focal length of 75 mm and f/1.4, and an output lens focal length of 85 mm and f/1.4. The image quality of this spectrograph is sufficient to image each 50 μm diameter fiber on a 2×2 pixel (about 54×54 μm) region of the CCD. Note that because the input and output focal lengths are not the same, the spectrograph has a magnification of 1.13, which restricts the number of FIC fibers that may be simultaneously detected to about 80 (representing a rectangular 8×10 fiber region at the collection end of the FIC fiber bundle). Larger spectral images are obtained simply by raster-scanning the sample over an array of adjacent rectangular regions, and concatenating the resulting single-frame images to form a spectral image of an arbitrarily large area. An N×N image is assembled from N×N×80 pixels; each pixel is in fact a 900 channel wide Raman spectrum, the Raman shifts window is from 100 cm⁻¹ to 1900 cm⁻¹. A review of all the remaining components of the NIRIM instrument, including mirrors, lenses, holographic filter, excitation fiber set up and other design considerations are set forth in A. D. Gift, J. Ma, K. S. Haber, B. L. McClain, D. Ben-Amotz, J. Raman Spectrosc. 1999, 30, 757-765, which was previously incorporated herein by reference.

In order to demonstrate that solid support matrices can be identified based upon their polymeric constituents regardless of the chemical nature of the molecule they are attached to (e.g. an amino acid oligomer) the following Merrifield resin beads carrying various protected amino-acids were positioned in the field of view of the NIRIM and single-bead Raman spectra were recorded between 100 cm⁻¹ and 1900 cm⁻¹: Boc-Ala-O-Merrifield (0.9 mmolg⁻¹); Boc-Asn-O-Merrifield (0.6 mmolg⁻¹); Boc-Asp(OBzl)-O-Merrifield (1 mmolg⁻¹); Boc-Cys(Acm)-O-Merrifield (0.8 mmolg⁻¹); Boc-Gln-O-Merrifield (0.6 mmolg⁻¹); Boc-Glu(OBzl)-O-Merrifield (0.8 mmolg⁻¹); Boc-His(DNP)-O-Merrifield (0.6 mmolg⁻¹); Boc-Ile-O-Merrifield (0.9 mmolg⁻¹); Boc-Leu-O-Merrifield (1 mmolg⁻¹); Boc-Lys(2-Cl-Z)-O-Merrifield (0.5 mmolg⁻¹); Boc-Met-O-Merrifield (0.9 mmolg⁻¹); Boc-Phe-O-Merrifield (0.8 mmolg⁻¹); Boc-Pro-O-Merrifield (0.9 mmolg⁻¹); Boc-Ser(OBzl)-O-Merrifield (0.6 mmolg⁻¹); Boc-Thr(OBzl)-O-Merrifield (0.6 mmolg⁻¹); Boc-Trp-O-Merrifield (0.6 mmolg⁻¹); Boc-Tyr(2-Br-Z)-O-Merrifield (0.6 mmolg⁻¹); Boc-Val-O-Merrifield (0.8 mmolg⁻¹). The unsubstituted beads studied utilizing the above described spectrographic technique are: TentaGel—S—OH (130 μm), 0.3 mmolg⁻¹; and Hydroxymethyl-polystyrene (˜90 μm), 1.1 mmolg⁻¹, 1% cross-linked (DVB/PS). Note that the above listed beads can be utilized in the present invention and are commercially available from Advanced ChemTech located in Louisville, Ky.

In particular, the aforementioned bead samples were placed on a sapphire single crystal (HEMEX (white), Crystal Systems, c-axis cut to eliminate fluorescence emission) positioned in the field of view of the NIRIM and images were recorded. The software used to either acquire or process the experimental data on the NIRIM instrument are pls_image.vi (data acquisition; system software written in LabView 4.1 (National Instruments)), nirim.vi (3-D data cube acquisition; system software written in LabView 4.1 (National Instruments)) and MultiSpec (spectral imaging analysis and classification; L. Biehl, D. Langrebe, “MultiSpec—A Tool for Multispectral Image Data Analysis”, Pecora 13, Sioux Falls, S.D., August 1996. The software is publicly available from Purdue University, West Lafayette, Ind., and can be downloaded at: http://dynamo.ecn.purdue.edu/˜biehl/multispecl, and is hereby incorporated herein by reference). The latter program requires the user to first select known regions of the image and identify their composition (training fields). The program then uses built-in algorithms (operator's choice) to statistically determine the most likely chemical identity for each fiber's Raman output in the image. The image is then redisplayed with the fibers' Raman output color-coded as to their most likely chemical identity. In this example the images were analyzed using the spectral angle mapping (SAM) algorithm, and the training fields were those of authentic samples of the beads.

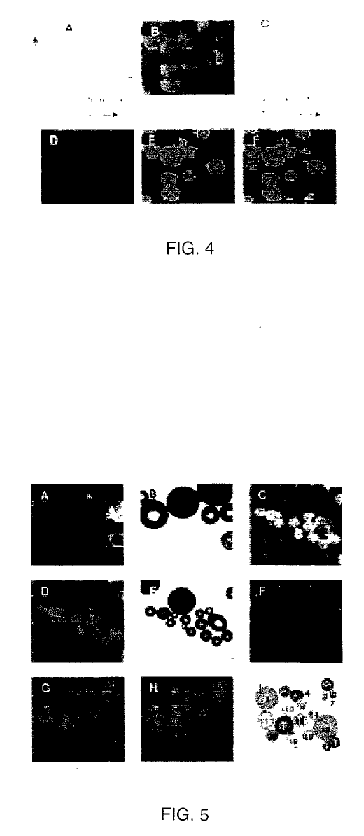

Visual inspection of these spectra indicated that the spectral features were dominated by the solid support matrix (polystyrene; PS) and even background subtraction (unsubstituted polystyrene beads) did not reveal the spectral features of the material attached to the beads. Hence, Raman imaging of PS supported compounds is insensitive to the material coupled to the beads at least up to 1.0 mmol of the amino-acids studied per gram of resin. Interestingly, while the spectral features of the attached material become detectable when present at a much higher amount, the vibrations of the solid support matrix and their intensity remain essentially unaffected. An extreme illustration of this result is that of TentaGel beads, which are 30/70 (w/w) PS/polyethyleneglycol graft copolymer. As shown by a comparison of panels A and C of FIG. 4, the Raman spectrum of TentaGel shows vibrations specific to the polyethyleneglycol (PEG) component as well as the fingerprint transitions of polystyrene (PS) which have not been affected by the PEG component of the resin.

In addition, the Raman spectra of polystyrene (PS) and TentaGel-S—OH beads displayed unique vibrations that were used to image and identify them selectively. Panel B of FIG. 4 shows a 5×5 FIC frames Raman image (50×40 FIC fibers and 15×12 μm per single image-pixel) of a mixture of PS and TentaGel-S—OH beads recorded with a 10× objective, a global illumination laser power of ≈400 mW per single frame region and a single frame detector integration time of 45 seconds (exposure time of 45 seconds per frame, total scan time 20 minutes; note that this acquisition time could be decreased by at least an order of magnitude with a more powerful laser source and a more efficient optics set-up). The classified images of TentaGel-S—OH beads (panel D of FIG. 4), polystyrene beads (panel F of FIG. 4), and a mixture of both TentaGel-S—OH beads and polystyrene beads (panel E of FIG. 4) were readily derived from the Raman data (panel B of FIG. 4) using the spectral angle mapper (SAM) algorithm of the spectral imaging software package MultiSpec which, as previously mentioned, is publicly available from Purdue University, West Lafayette, Ind., and can be downloaded at: http://dynamo.ecn.purdue.edu/˜biehl/multispec/. Transitions at 1277 cm⁻¹ for TentaGel-S—OH (panel D of FIG. 4) and at 1000 cm⁻¹ and 1031 cm⁻¹ for PS (panel F of FIG. 4) were used to identify the corresponding beads. Note that panel E of FIG. 4 an image where both PS and TentaGel beads were specifically and concomitantly identified.

To further demonstrate the ability to reliably identify a solid support matrix (e.g. resin beads) based on unique differences in their chemical nature, the following PS and non-PS based beads were subjected to the above described spectrographic technique: (a) 4-Bromo-PS 200-400 mesh (2.5 mmolg⁻¹, commercially available from Chem-Impex International located in Wood Dale, Ill.); (b) 4-Carboxy-PS 100-200 mesh (3.5 mmolg⁻¹, commercially available from Novabiochem); (c) PEG cross-linked PS 100-200 mesh (2 mmolg⁻¹, commercially available from Advanced ChemTech); (d) Amino-PEGA (0.4 mmolg⁻¹, commercially available from Novabiochem, located Läufelfingen, Switzerland); (e) HMBA-SPAR 50 100-200 mesh (polyacrylamide resin, 0.3 mmolg⁻¹, commercially available from Advanced ChemTech); and (f) SPAR 50 200-400 mesh (0.8 mmolg⁻¹, commercially available from Advanced ChemTech). The aforementioned beads can also be utilized in the present invention. Beads a-c were chosen to establish that at least 3 additional PS based beads can be readily distinguished (FIG. 5 panel A). Beads d-f were chosen to establish the same conclusion for polyamide based beads (FIG. 5 panel F), and to demonstrate that they can also be readily differentiated from PS-based beads (FIG. 5 panels C, D, and panels F-H).

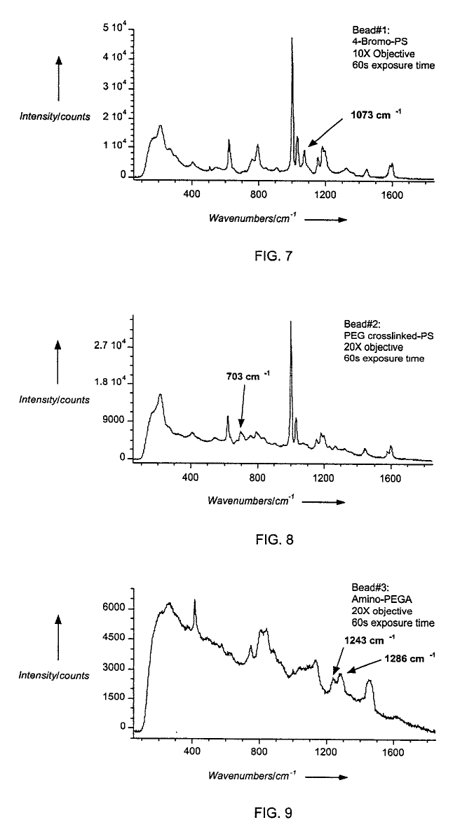

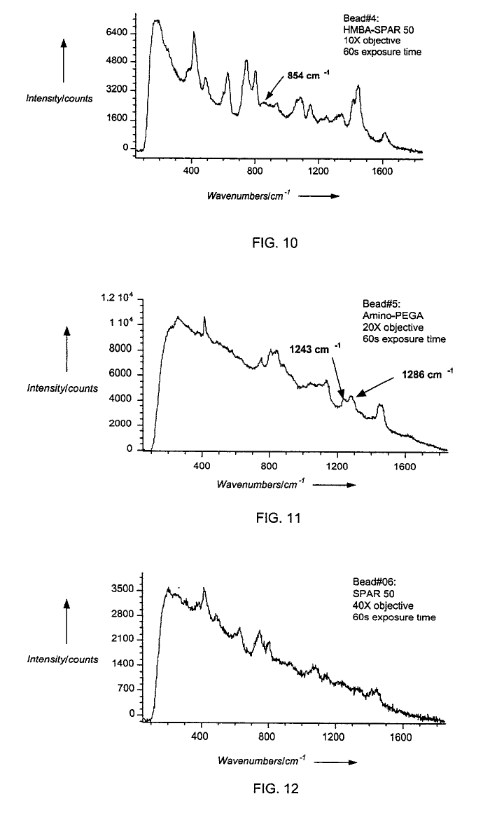

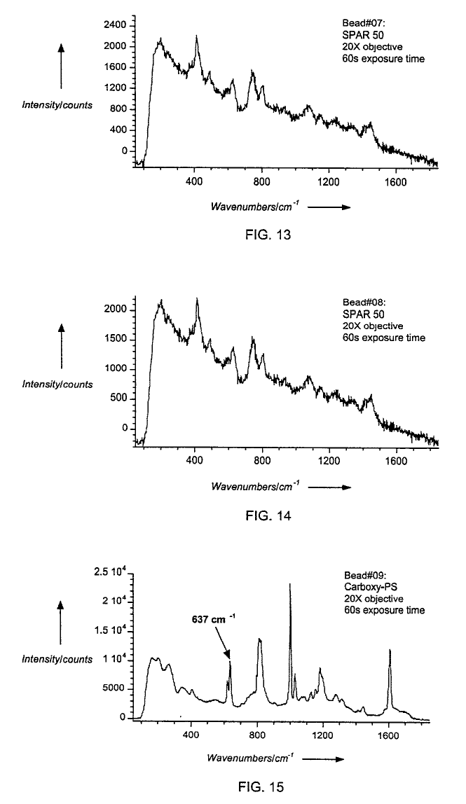

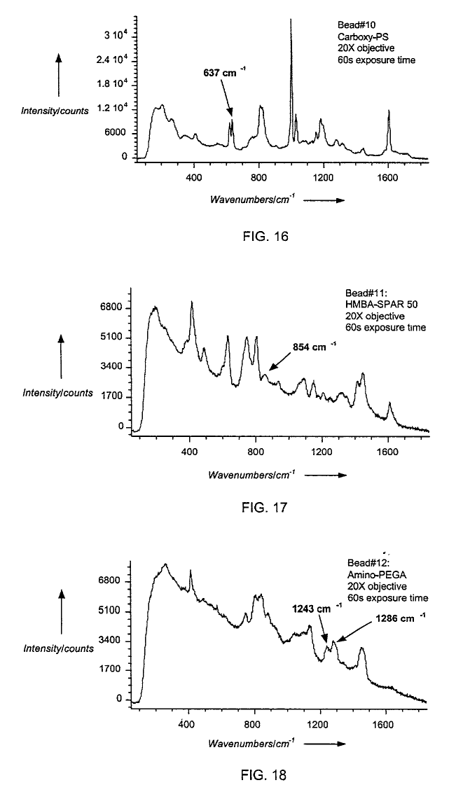

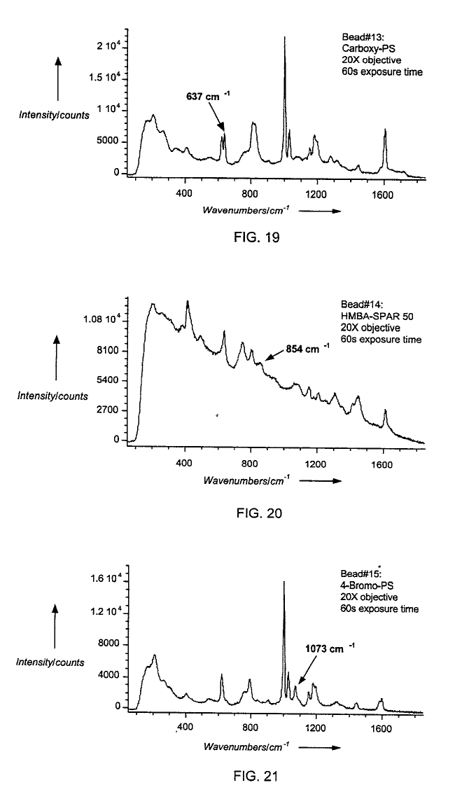

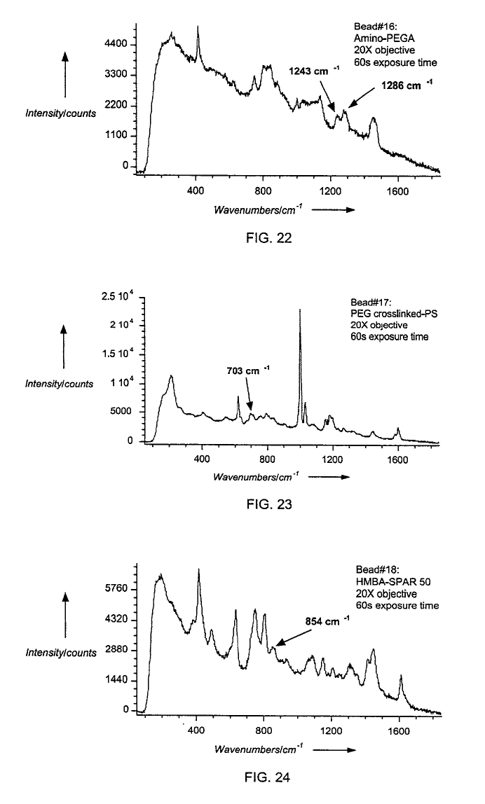

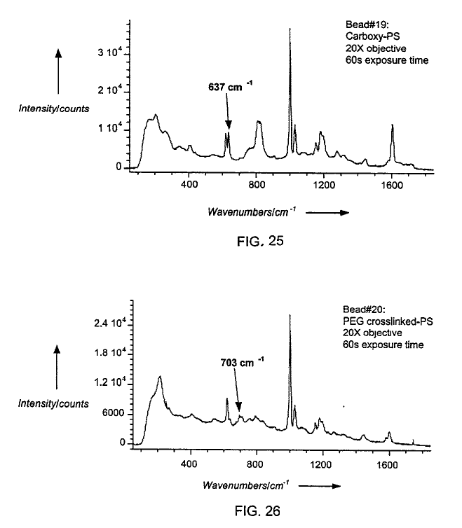

One mg each of the aforementioned beads were combined in methanol so as to produce a statistical mixture of the six different beads, i.e. (a) 4-Bromo-PS, (b) 4-Carboxy-PS, (c) PEG cross-linked PS, (d) Amino-PEGA, (e) HMBA-SPAR 50, and (f) SPAR 50. A drop of this mixture of beads was deposited on a sapphire. After evaporation of the methanol the sapphire was placed in the field of view of the NIRIM. A library of single bead near IR-Raman spectra of each of the beads was first recorded, then several regions were arbitrarily selected for multispectral imaging. FIG. 5 shows 5×5 frames (50×40 pixels) and 6×6 frames (60×48 pixels) in which all the beads were identified following the same procedure as discussed in reference to FIG. 4. Panel A of FIG. 5 shows specific NIR-Raman imaging of 4-bromo-PS (blue, 1073 cm⁻¹), 4-carboxy-PS (green, 637 cm⁻¹), and PEG cross-linked PS (red, 703 cm⁻¹). Panel B of FIG. 5 shows a white-light image of the beads in panel A. Panel C of FIG. 5 shows specific NIR-Raman imaging of 4-bromo-PS (blue), 4-carboxy PS (green), and HMBA-SPAR 50 (red, 854 cm⁻¹). Panel D of FIG. 5 shows the same image as in panel C, but only the beads with a PS backbone are visualized. In addition, the two PS-based beads were color coded using vibrations specific to each of them (4-bromo-PS, blue, 4-carboxy-PS green). Panel E of FIG. 5 shows a white light image of the beads shown in panels C and D. Panel G of FIG. 5 shows specific NIR-Raman imaging of polyamide based beads (green, amino-PEGA, HMBA-SPAR 50, SPAR 50). Panel F of FIG. 5 shows specific near IR-Raman imaging of PS beads (red 4-bromo-PS, 4-carboxy-PS, PEG cross-linked-PS). Panel H of FIG. 5 shows an NIR-Raman image where PS-and polyamide-based beads were selectively and concomitantly identified. As further discussed below, panel I of FIG. 5 is a white light image of the beads shown in panels F-H. Since each bead is a collection of pixels and each pixel is a near IR-Raman spectrum of that area of the bead, comparison of these pixel-spectra with the library of single-bead spectra recorded on the authentic samples (see FIGS. 7-26) confirmed the automated assignments. These results were reproducible regardless of the size and shape of the beads.

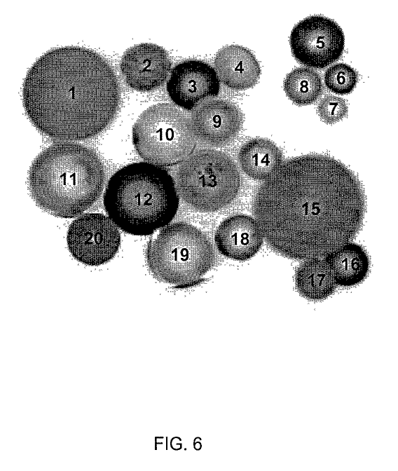

FIG. 6 is an enlarged view of panel I of FIG. 5. In particular, FIG. 6 shows a white light image of the beads shown in panels F-H of FIG. 5. The beads were identified by multispectral imaging as discussed above, in addition the beads were identified by single bead microspectroscopy. In particular, the number on each bead refers to the single bead near IR-Raman spectra which are set forth in FIGS. 7-26 (i.e. 4-bromo-PS: beads No. 1 and 15; 4-carboxy-PS: beads No. 9, 10, 13; PEG cross-linked-PS: beads No. 2, 17, 20; amino-PEGA: beads No. 3, 5, 12, 16; HMBA-SPAR50: beads No.4, 11, 14, 18; SPAR 50: beads No.6,7,8).

It should be appreciated that the identification of the first randomized position of a compound attached to a solid support matrix using the present invention has been demonstrated, e.g. using near IR-Raman imaging of self-encoded resin beads. However, it should be understood that any imaging technique could be applicable to the method of the present invention as long as the solid support matrix used displays unique spectral features, and provided the compounds (e.g. amino acid oligomers) attached to the solid support matrix does not significantly alter their spectral signature. For instance, secondary ion mass spectrometry (SIMS) and FTIR imaging are alternative approaches which may be used in the present invention.

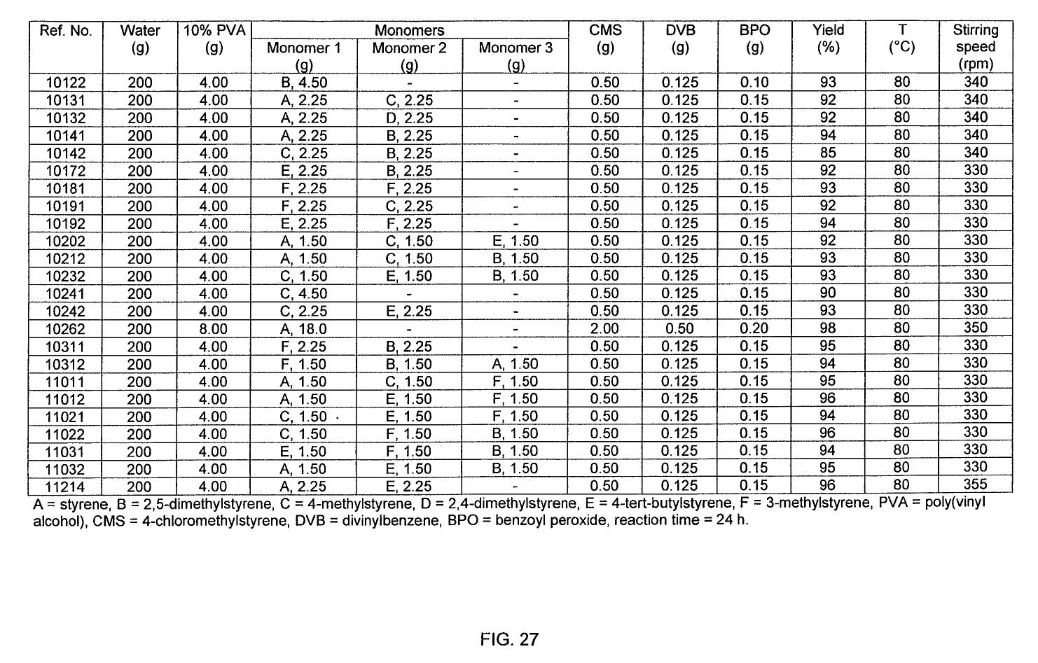

Furthermore, other solid support matrices other than the ones specifically mentioned above can be utilized in the present invention as long as they have a spectroscopically distinct chemical group. For example, various chemically distinct beads of polystyrene resin from 1% divinylbenzene/stryrene doped with spectroscopically detectable amounts and combinations of Raman distinguishable para-substituted styrene monomers (e.g. substituants include —CN, —OCH₃, —F, —Cl, Br, —I, —CH₃, —C₆H₅, —NO₂, —Si(CH₃)₃, and —SO₂CH₃) can be utilized in the present invention as the solid support matrix. The following procedure was utilized to produce specific examples of solid support matrices in the form of beads which can be employed in the present invention. The micro-spherical beads were prepared by suspension copolymerization using water as the continuous phase. In particular, 200 mL of deionized water, 4 g of 10% (wt.) poly(vinylalcohol) (PVA) solution were placed in an Arshady vessel (Arshady, R.; Ledwith, A., Suspension Polymerization and its application to the preparation of polymer supports, Reactive Polymers 1983, 1, 159-174, incorporated herein by reference) equipped with a mechanical stirrer, condenser, and N₂ inlet. The reaction vessel was kept under nitrogen atmosphere throughout the polymerization process. An organic solution composed of 1.5 g of styrene, 1.5 g of 4-methylstyrene, 4-tert-butylstyrene, 0.125 g of divinyl benzene (DVB), 0.5 g of chloromethylstyrene (CMS), 0.15 g of benzoyl peroxide (BPO) was added to the reaction vessel. Benzoyl peroxide (BPO), poly(vinyl alcohol) (PVA), divinyl benzene (80%) and all polymers are commercially available from Aldrich Inc., located in St. Louis, Mo. (Note that the monomers were distilled under reduced pressure to remove the inhibitors and stored under refrigeration until use.) The mixture was stirred at a fixed speed of 330 rpm to produce the desired droplets size, hence the desired bead size. The reactor was immersed in a preheated oil bath maintained at 80° C. After 24 h, the motor was stopped and the newly formed beads were filtered and washed with deionized water. The beads were then extracted with water and ethanol using a Soxhlet extractor (24 h each). The beads were then sieved and dried under vacuum and characterized by FTIR and Raman in the above described manner. Yield of polymerization, 92%. FIG. 27 summarizes the conditions for bead synthesis.

Note that during the polymerization, microdroplets might coagulate together as their viscosity increases. Therefore, as indicated above, the aqueous phase is charged with a stabilizer, usually a water-soluble polymer. There are several polymers which can be used as the stabilizer, such as PVA (see above), gelatin, methyl cellulose, poly(methacrylic acid), and poly(vinyl pyridone). The choice of stabilizer may depend upon which monomers are being utilized. Moreover, selection of the appropriate stabilizer facilitates appropriate bead formation. The appropriate stabilizer can be determined by routine experimentation. Also note that the size of the beads is dependent upon the size of the microdroplets. The parameters effecting microdroplet size include reactor design, the rate of mixing (stirring), ratio of the monomer phase to the aqueous solution, viscosity of both phase, and type and concentration of the droplet stabilizer. Adjusting the stirring speed provides the most convenient way to control the bead size.

While the invention has been illustrated and described in detail in the drawings and foregoing description, such illustration and description is to be considered as exemplary and not restrictive in character, it being understood that only the preferred embodiments have been shown and described and that all changes and modifications that come within the spirit of the invention are desired to be protected.

What is claimed is:

1. A method for determining the primary structure of a first compound which is bound to a first solid support matrix, comprising:
(a) reacting a first building block of said first compound with said first solid support matrix so that said first building block is bound to said first solid support matrix;
(b) subjecting said first solid support matrix to a spectroscopic technique so as to generate spectrographic data of said first solid support matrix;
(c) determining a chemical composition of said first solid support matrix said first building block is bound to based upon said data generated by said spectrographic technique; and
(d) determining the chemical identity of said first building block based upon the chemical composition of said first solid support matrix.

2. The method of claim 1, further comprising:
(e) reacting a second building block of said first compound with said first building block so that said second building block is covalently bound to said first building block.

3. The method of claim 1, further comprising:
(f) reacting a first building block of a second compound with a second solid support matrix so that said first building block of said second compound is bound to said second solid support matrix, said second compound having a primary structure which is different from said primary structure of said first compound;
(g) subjecting said second solid support matrix to said spectroscopic technique so as to generate spectrographic data;
(h) determining the chemical composition of said second solid support matrix said first building block of said second compound is bound to based upon said data generated by said spectrographic technique, said second solid support matrix having a chemical composition which is different from said chemical composition of said first solid support matrix; and
(i) determining the chemical identity of said first building block of said second compound based upon the chemical composition of said second solid support matrix, said chemical identity of said first building block of said second compound being different from said chemical identity of said first building block of said first compound.

4. The method of claim 1, wherein:
said spectroscopic technique includes a Raman spectroscopic technique.

5. The method of claim 1, wherein:
said first compound is an amino acid oligomer.

6. The method of claim 1, wherein:
said first solid support matrix is configured as a bead.

7. The method of claim 6, wherein:
said bead is selected from a group of beads consisting of Merrifield, Tenta Gel, 4-bromo-polystyrene, 4-carboxy-polystyrene, a PEG cross-linked Merrifield, Amino-PEGA, HMBA-Spar 50, and SPAR 50.

8. The method of claim 1, further comprising:
(j) screening said first compound prior to (b) so as to determine whether said first compound possess a predetermined characteristic.

9. The method of claim 8, wherein:
(j) includes determining whether said first compound binds to a receptor.

10. The method of claim 1, wherein:
(a) includes generating a combinatorial library prior to (b), said combinatorial library includes (i) said first compound bound to said first solid support matrix and (ii) a second compound bound to a second solid support matrix, wherein (i) said primary structure of said first compound is different from the primary structure of said second compound and (ii) said second solid support matrix has a chemical composition which is spectroscopically distinct from said chemical composition of said first solid support matrix.

11. A method of screening a combinatorial library which includes (i) a first solid support matrix, (ii) a second solid support matrix, (iii) a first compound having a building block thereof directly chemically bound to said first solid support matrix, and (iv) a second compound having a building block thereof directly chemically bound to said second solid support matrix, wherein (i) said first compound has a primary structure which is different from the primary structure of said second compound and (ii) said first solid support matrix has a chemical composition which is spectroscopically distinct from a chemical composition of said second solid support matrix, comprising:
(a) subjecting said first solid support matrix to a spectroscopic technique so as to generate spectrographic data of said first solid support matrix;
(b) utilizing said spectrographic data to distinguish said first solid support matrix from said second solid support matrix; and
(c) determining the chemical identity of said building block of said first compound which is directly chemically bound to said first solid support matrix based upon said spectroscopically distinct chemical composition of said first solid support matrix.

12. The method of claim 11, further comprising:
(d) subjecting said first compound to a deconvolution process after (a).

13. The method of claim 11, wherein:
said spectroscopic technique includes a Raman spectroscopic technique.

14. The method of claim 11, wherein:
said first compound is an amino acid oligomer.

15. The method of claim 1, wherein:
said first solid support matrix is configured as a bead.

16. The method of claim 15, wherein:
said bead is selected from a group of beads consisting of Merrifield, Tenta Gel, 4-bromo-polystyrene, 4-carboxy-polystyrene, a PEG cross-linked Merrifield, Amino-PEGA, HMBA-Spar 50, and SPAR 50.

17. The method of claim 11, further comprising:
(e) screening said first compound prior to (b) so as to determine whether said first compound possess a predetermined characteristic.

18. The method of claim 8, wherein:
(e) includes determining whether said first compound binds to a receptor.

19. A method of screening a combinatorial library which includes (i) a first bead, (ii) a second bead, (iii) a first amino acid oligomer having an amino acid located in a first position, said amino acid located in said first position being directly chemically bound to said first bead, and (iv) a second amino acid oligomer chemically bound to said second bead, wherein (i) said first amino acid oligomer has a primary structure which is different from the primary structure of said second amino acid oligomer and (ii) said first bead has a chemical composition which is spectroscopically distinct from a chemical composition of said second bead, comprising:
(a) subjecting said first bead to a spectroscopic technique so as to generate spectrographic data of said first bead;
(b) utilizing said spectrographic data to distinguish said first bead from said second bead; and
(c) determining the chemical identity of said amino acid of said first amino acid oligomer which is located in said first position based upon said spectroscopically distinct chemical composition of said first bead.

20. The method of claim 19, further comprising:
(d) subjecting said first amino acid oligomer to a deconvolution process after (a).

21. The method of claim 19, wherein:
said spectroscopic technique includes a Raman spectroscopic technique.

22. The method of claim 19, wherein:
said first bead is selected from a group of beads consisting of Merrifield, Tenta Gel, 4-bromo-polystyrene, 4-carboxy-polystyrene, a PEG cross-linked Merrifield, Amino-PEGA, HMBA-Spar 50, and SPAR 50.

23. The method of claim 19, further comprising:
(e) screening said first amino acid oligomer prior to (b) so as to determine whether said first amino acid oligomer possess a predetermined characteristic.

24. The method of claim 23, wherein:
(e) includes determining whether said first amino acid oligomer binds to a receptor.
